(19) United States
(12) Patent Application Publication
(10) Pub. No.: US 2010/0203126 A1
(43) Pub. Date: Aug. 12, 2010

(54) MULTILAYERED VITAMIN COMPLEX TABLET CONTAINING UBIDECARENONE

(76) Inventors: Jong-Woo Park, Gyeonggi-do (KR); Jin-Woo Han, Gyeonggi-do (KR); Yae-Young Choi, Seoul (KR)

Correspondence Address: DICKSTEIN SHAPIRO LLP, 1825 EYE STREET NW, Washington, DC, 20006-5403, US

(21) Appl. No.: 12/668,328

(22) PCT Filed: Jul. 11, 2008
(86) PCT No.: PCT/KR08/04111

§ 371 Date: Jan. 8, 2010

(30) Foreign Application Priority Data
Jul. 11, 2007 (KR) 10-2007-0069558

(51) Int. Cl. A61K 9/20; A61K 31/341; A61K 31/355; A61K 31/23; A61K 31/7056; A61K 31/4406; A61K 31/675; A61K 31/122; A61K 33/30; A61K 33/26; A61K 33/42; A61K 9/28

(52) U.S. Cl. 424/465; 514/474; 424/464; 514/458; 514/552; 514/52; 514/355; 514/89; 514/678; 424/643; 424/646; 424/602; 424/474

(57) ABSTRACT

The present invention relates to a multi-layered vitamin/mineral complex tablet having enhanced stability of ubidecarenone. The present invention is characterized in that ubidecarenone is contained in a first layer, and ingredients decreasing the stability of ubidecarenone are contained in an additional layer separated from the first layer. The method is a convenient process, and the formulation prepared by the method can maintain a high content of ubidecarenone during long-term storage, thereby providing a simultaneous intake of ubidecarenone and nutritional ingredients such as various vitamins.

### TECHNICAL FIELD

The present invention relates to a vitamin complex tablet containing ubidecarenone.

### BACKGROUND ART

Ubidecarenone (Coenzyme Q₁₀; molecular formula C₅₉H₉₀O₄; molecular weight 863.36) is one of coenzyme synthesized by the human body, and functions as a transmitter component in the electron transfer system through repeated oxidation and reduction in mitochondria. Generally, about 40 to 90% thereof occurs in reduced form in living bodies, and the reduced ubidecarenone is known to have antioxidant effect.

Examples of the physiological functions of ubidecarenone include the activation of energy production through activating mitochondrial function, activation of cardiopulmonary function, stabilization of cellular membranes, production of cells through an anti-oxidative effect, myocardial protection action, prevention of carcinogenesis, anti-aging action, and LDL oxidation suppression in blood, suppression of elevation in blood pressure, and amelioration in oxygen utilization efficiency in myocardial ischemia. In medical fields, ubidecarenone is used as a metabolic cardiotonic drug, and applied for the treatment of diseases such as heart failure, aging, arteriosclerosis, diabetes, hypertension, hypotension, angina pectoris, ischaemic heart disease, and degenerative muscular atrophy.

Ubidecarenone is generally found in fish, meat, or seaweed, but rarely in natural foods. Thus, since ubidecarenone cannot be sufficiently taken by typical meals, it is commercially available as a nutrient additive or drug.

In particular, ubidecarenone directly removes oxygen free radical which causes cancer, adult diseases, or aging, and facilitates functions of other antioxidants (vitamin C, E), and thus it is usually taken along with other antioxidant vitamins. When ubidecarenone is administered with other antioxidants including beta-carotene, selenium, vitamin C, and vitamin E, the efficacy is increased to 2-3 fold or more than that of its single administration. It has been reported that the coexistence of ubidecarenone is very important for vitamin E to exert its antioxidative activity.

Ubidecarenone is an oil-soluble substance, and thus is insoluble in water and alcohol, but highly soluble in ether. In addition, ubidecarenone is very unstable and degrades to produce hydroquinone when it reacts with light, air, or other vitamins. Therefore, in order to increase the uptake of ubidecarenone, it is needed to increase its solubility and stability. Generally, it is taken in a form of a stabilized tablet or capsule, or a solubilized and/or stabilized liquid formulation with lipids.

Its oral liquid formulations having enhanced solubility and bioavailability are disclosed in U.S. Pat. No. 5,035,895, U.S. Pat. No. 6,300,377, U.S. Pat. No. 6,441,050, U.S. Pat. No. 6,652,891, JP P2003-169630A, and JP P2005-43A. Specifically, GB 1112568 discloses that the liquid formulation is stabilized by using a suspending agent and a non-ionic surfactant, U.S. Pat. No. 7,026,361 discloses that its stabilization and bioavailability is improved by using a water-soluble polymer, an emulsifier, and a suspending agent.

JP P2004-16011A discloses that a drink, which contains ubidecarenone stabilized by using one or more sugar alcohols and organic acids, and JP P2003-169630A discloses that dispersion and stability of ubidecarenone and other vitamins are improved by using sodium caseinate and dextrin. JP P2004-81158A discloses that coenzyme Q is emulsified in an aqueous solution, and organic acids are added thereto to increase its stability.

On the other hand, U.S. Pat. No. 5,443,842, U.S. Pat. No. 6,740,338, and U.S. Pat. No. 6,855,733 disclose that soft or hard oral capsules, filled into gelatin capsule. In particular, U.S. Pat. No. 6,995,820 and JP P2001-354553A disclose that ubidecarenone stabilized in capsule is disclosed in. When ubidecarenone is present with gelatin, its degradation is generally accelerated due to contact with soluble vitamins. Therefore, organic acids, mixtures thereof, and other stabilizers are used in the above references. JP 2005-124482 discloses a nutrient composition containing beta carotene and enzyme treated rutin.

All of the above references disclose that the stability and bioavailability of ubidecarenone are enhanced by formulations and additives. However, there is still a need for solid tablet formulations, in which they contain various vitamins with minimized amount of other excipients, and thus their size is small and various vitamins and minerals and ubidecarenone can be taken all at once, as an alternative of suspended liquids or big-sized capsules.

Meanwhile, JP P2005-2005A discloses stabilization techniques for treating ubidecarenone itself have been studied, and exemplified by encapsulation with cyclodextrin, and KP2002-0080370A discloses a technique for enhancing the solubility and stability by complex formation of ubidecarenone and gamma cyclodextrin.

JP 2006-182770A discloses a preparation method of solid formulations, in which one group containing ubidecarenone along with organic acid as stabilizer, and the other group containing vitamin B1 are granulated separately, and then mixed together to improve the stability of ubidecarenone. However, the separation of ubidecarenone and vitamin B1 by the granulation process has a problem that of large contact area, which leads to low stability. Accordingly, the stability can be enhanced by using organic acids that is known to suppress the degradation of ubidecarenone.

As described above, the known techniques of stabilizing ubidecarenone generally require either an additional process of directly treating ubidecarenone, or stabilizers such as organic acids as an essential component.

Problematically, organic acids are expensive, and artificial use thereof causes blood acidification whichthus may generate head ache, dizziness, insomnia, fatigue or the like. In addition, organic acids stimulate intestinal secretion, causing diarrhea. Organic acids such as oxalate and phytate bind with calcium in the digestive organs to produce insoluble salts, thereby inhibiting calcium absorption.

Furthermore, when organic acids co-exist with unstable vitamins and minerals under acidic conditions, such as vitamin K, folic acid, vitamin A, etc., they may increase the risk of reducing their stability.

In the preparation of single formulations containing nutritional ingredients such as ubidecarenone, vitamins and minerals, the prior arts are limited to vitamins such as vitamin B and E. There is no mention of the stability of ubidecarenone with respect to other vitamins and minerals that are contained in the typical vitamin complex tablet.

Accordingly, the present inventors have examined other nutritional ingredients (vitamins, minerals, amino acids, etc.) decreasing the stability of ubidecarenone under the co-existence, and they have developed a multi-layered tablet, in which a layer containing the nutritional ingredients and other layer containing ubidecarenone are separated, and thus ubidecarenone is stably separated and stored in one solid formulation without an additional complex process or addition of stabilizers such as organic acids, thereby completing the present invention.

### DISCLOSURE OF INVENTION

### Technical Problem

In order to overcome the limitations of the prior art, the present invention provides a vitamin complex tablet comprising both ubidecarenone and nutritional ingredients of various vitamins, in which the vitamin complex tablet is a multi-layered oral vitamin complex tablet containing ubidecarenone, prepared by a simple and inexpensive process, capable of maintaining a high content of ubidecarenone during long-term storage.

### Technical Solution

In order to overcome the limitations of the prior art, it is an object of the present invention to provide a vitamin complex tablet comprising both ubidecarenone and nutritional ingredients such as various vitamins and minerals, in which the vitamin complex tablet is a multi-layered oral vitamin/mineral complex tablet containing ubidecarenone, prepared by a simple and inexpensive process, capable of maintaining a high content of ubidecarenone during long-term storage.

Further, it is another object of the present invention to provide a multi-layered oral vitamin/mineral complex tablet containing ubidecarenone, capable of maintaining the stability of ubidecarenone for a long period time, even though it comprises an excessive amount of vitamin, mineral or the like.

### Advantageous Effects

The present invention is characterized in that ubidecarenone is contained in a first layer, and other ingredients decreasing the stability of ubidecarenone are contained in an additional layer being separated from the first layer. The method is convenient and the formulation prepared by the method can maintain a high content of ubidecarenone during long-term storage, thereby providing a simultaneous intake of ubidecarenone and nutritional ingredients such as various vitamins and minerals.

### BEST MODE FOR CARRYING OUT THE INVENTION

In order to achieve the above objects, the present invention provides an ubidecarenone-containing multi-layered vitamin complex tablet, characterized in that it is a multi-layered tablet comprising a first layer containing ubidecarenone (first ingredient) and a separated second layer containing an active ingredient (second ingredient) decreasing the stability of ubidecarenone, the first ingredient and the second ingredient are not simultaneously present in the same layer, and the second ingredient contains a vitamin selected from ascorbic acid, alpha-tocopherol acetate, retinol palmitate, cyanocobalamine, nicotinamide, pyridoxine hydrochloride, chole-calciferol, fursultiamine, inositol, derivatives thereof and mixtures thereof.

Further, the present invention provides an ubidecarenone-containing multi-layered vitamin complex tablet, characterized by maintaining at 90% or more of the content of ubidecarenone upon long-term storage stability test (24 months: 25±2° C., 60±5% RH) or accelerated stability test (6 months: 40±2° C., 75±5% RH).

Further, the present invention provides an ubidecarenone-containing multi-layered vitamin complex tablet, characterized in that the second layer further contains a mineral selected from zinc sulfate, iron sulfate, choline bitartrate, calcium hydrogen phosphate and mixtures thereof, amino acids selected from L-cysteine, derivatives thereof and mixtures thereof, and herbal extracts selected from gamma-oryzanol, derivative thereof and mixtures thereof.

Further, the present invention provides an ubidecarenone-containing multi-layered vitamin complex tablet, characterized in that the first and second layers have a structure of upper/lower layers or inner/outer layers.

Further, the present invention provides an ubidecarenone-containing multi-layered vitamin complex tablet, characterized in that the complex tablet is in a form of three-layered tablet, where the second layer is divided by at least two sublayers, so that the vitamin exists in one sublayer and the mineral exists in the other sublayer.

Further, the present invention provides an ubidecarenone-containing multi-layered vitamin complex tablet, characterized by further comprising at least one excipients, disintegrants, binders, glidants and sweetening agents.

Further, the present invention provides an ubidecarenone-containing multi-layered vitamin complex tablet, characterized in that at least one moisture-proof coating and light-shielding coating are applied to the complex tablet.

Further, the present invention provides a method of preparing the ubidecarenone-containing multi-layered vitamin complex tablet, characterized in that one of the raw materials for the first layer containing ubidecarenone (as a first ingredient) and for the second layer containing at least one active ingredient (as a second ingredient) that decrease the stability of ubidecarenone is subjected to compression, and then the other raw material is subjected to compression on to the above compressed raw material in a form of upper/lower layers or inner/outer layers, so as to obtain a multi-layered tablet. The second ingredient contains a vitamin selected from ascorbic acid, alpha-tocopherol acetate, fursultiamine, retinol palmitate, cyanocobalamine, nicotinamide, pyridoxine hydrochloride, cholecalciferol, inositol, derivatives thereof and mixtures thereof, and the first and second ingredients are not simultaneously present in the same layer.

Hereinafter, the present invention will be described in more detail.

The multi-layered tablet of the present invention is a multi-layered vitamin complex tablet comprising ubidecarenone in a first layer and the active ingredients decreasing the stability of ubidecarenone such as minerals and vitamins in other separated layer. The present invention provides a convenient process, which does not require addition of stabilizer improving the stability of each raw material, and pretreatment such as shielding and encapsulation, and also provides a convenient tablet formulation, which has a small size since it contains various vitamins with minimized amount of other excipients, and by which a patient can take various vitamins and minerals along with ubidecarenone at once, instead of suspended liquids or big-sized soft capsules.

The mixture of ubidecarenone and other vitamins/minerals may be prepared according to the typical methods, and the mixture may contain pharmaceutically acceptable excipients, disintegrants, binders, glidants, sweetening agents or the like, in addition to ubidecarenone and other vitamins/minerals. The multi-layered tablet prepared using the mixture may be coated with film or sugar.

The layer containing ubidecarenone of the present invention may include ubidecarenone, ubidecarenone derivatives and ingredients that do not decrease the stability of ubidecarenone under the co-existence, and ingredients decreasing the stability of ubidecarenone may be used, as long as they do not inhibit the stability of ubidecarenone in a formulation.

The ingredients of the layer containing vitamin and mineral may include orally administrable vitamins that inhibit the stability of ubidecarenone under the co-existence, such as vitamins including ascorbic acid, alpha-tocopherol acetate, fursultiamine, retinol palmitate, cyanocobalamine, nicotinamide, pyridoxine hydrochloride, chole-calciferol, and inositol, and derivatives thereof, orally administrable minerals that inhibit the stability of ubidecarenone under the co-existence, such as minerals including zinc sulfate, iron sulfate, choline bitartrate, and calcium hydrogen phosphate, and derivatives thereof, orally administrable amino acids and herbal extracts that inhibit the stability of ubidecarenone under the co-existence, such as minerals including L-cysteine and gamma-oryzanol, and derivatives thereof, or mixtures thereof.

The excipients used in the present invention may include at least one selected from the group consisting of orally administrable lactose, white sugar, glucose, fructose, mannitol, corn starch, potato starch, wheat starch, pregelatinized starch, micro-crystalline cellulose or cellulose derivatives, dextrin, calcium hydrogen phosphate, calcium phosphate monobasic, ethylcellulose, and methylcellulose.

The binders used in the present invention may include one or more selected from the group consisting of orally administrable polyvinylpyrrolidone or derivatives thereof, vinylpyrrolidone/vinylacetate copolymer, hydroxycellulose, methylcellulose, ethyl-cellulose, polyvinylalcohol, gelatin, corn starch, potato starch, wheat starch, pregelatinized starch, hydroxypropylmethylcellulose, and hydroxypropylcellulose.

The disintegrants used in the present invention may include at least one selected from the group consisting of orally administrable croscarmellose sodium, sodium starch glycolate, sodium carboxymethylcellulose, calcium carboxymethylcellulose, crospovidone, and etc.

The glidants used in the present invention may include at least one selected from the group consisting of orally administrable magnesium stearate, calcium stearate, sodium stearyl fumarate, stearic acid, talc, and silicone dioxide.

The sweetening agents used in the present invention may include at least one selected from the group consisting of acesulfame potassium, sucralose, aspartame, stevioside, and saccharin.

The coating agent used in the present invention is not specifically limited, and more preferably a coating agent having the functions of moisture-proof and light-shielding for the purpose of maintaining the stability of ubidecarenone and vitamin.

The coating base used in the coating agent include cellulose derivatives such as hydroxypropylcellulose, hydroxypropylmethylcellulose, ethylcellulose, hydroxypropyl-methylcellulose phthalate, hydroxypropylmethylcellulose acetate succinate, carboxymethylethylcellulose, and cellulose acetate phthalate, synthetic copolymers such as polyvinylacetaldiethylaminoacetate and aminoalkylmethacrylate copolymer E, methacrylic acid copolymer L or LD, aminoalkylacrylate copolymer RS, and polyvinylpyrrolidone, dextrin, fluran, zein, sodium alginate, white sugar, gelatin, and serac. Examples of the plasticizer include macrogol, trimethyl citrate, triacetin, medium-chain triglycerides, and glycerin. Examples of the glidants include talc, stearic acid, magnesium stearate, and sucrose esters of fatty acid, and examples of the light-shielding agent or coloring agent include metal oxide such as titanium oxide, yellow 3 ferric oxide, red 3 ferric oxide, and black 3 ferric oxide, and tar colors.

The pharmaceutical composition according to the present invention may be formulated into an orally administrable solid multi-layered tablet such as bilayered tablets, three-layered tablets, and core tablets.

The tableting method applied for the preparation of the multi-layered tablet may include any known methods. However, with respect to the multi-layered tablet, a pressure of 4.9 kN (kilo Newton, 10 ton=98 kN) or less is preferably applied upon first tableting. When a higher pressure is applied thereto, the tablet becomes too hard to bind with the second layer. Upon second tableting, a pressure of 1 to 2 ton, that is, 11.76 to 14.7 kN is applied.

### MODE FOR THE INVENTION

Hereinafter, the present invention will be described in more detail with reference to Examples. However, these Examples are for illustrative purposes only, and the invention is not intended to be limited by these Examples.

I. Examination of Ingredients Decreasing Stability of Ubidecarenone

In order to examine the ingredients decreasing the stability of ubidecarenone, monolayered tablets containing various ingredients of vitamin/mineral/herbal extract/amino acid and ubidecarenone were prepared (Preparation Examples 1-1 to 1-7), their stability was evaluated (Experimental Example 1). The detailed description is as follows.

1. Preparation of Ubidecarenone-Containing Monolayered Tablets by Various Formulation Methods

### (1) Preparation Example 1-1

### Formulation of Ubidecarenone-Containing Monolayered Tablet

TABLE 1

| Ingredient | Weight (per one tablet) | |
|---|---|---|
| Ubidecarenone | 5.0 | mg |
| Low-substituted hydroxymethyl cellulose | 20.0 | mg |
| Ludipress | 200.0 | mg |
| Povidone | 2.5 | mg |
| Magnesium stearate | 10.7 | mg |
| Talc | 1.7 | mg |

Ubidecarenone and low-substituted hydroxymethyl cellulose, ludipress, povidone were mixed for 10 minutes, and then magnesium stearate, talc, and silicone dioxide were added thereto. The mixture was blended for 5 minutes to prepare a powder. The mixed powder was filled into a lower punch below a die using a feeder. Then, compression was performed using an upper punch, and a tablet was discharged from the die to prepare an ubidecarenone-containing monolayered tablet.

### (2) Preparation Example 1-2

### Formulation of Ubidecarenone-Containing Monolayered Tablet

TABLE 2

| Ingredient | Weight (per one tablet) | |
|---|---|---|
| Calcium pantothenate | 30.0 | mg |
| Selenium (dried yeast) | 18.0 | mg |
| Ubidecarenone | 5.0 | mg |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |

Ubidecarenone and lactose were granulated using povidone as a binder, and then sieved. Selenium, calcium pantothenate, and low-substituted hydroxymethyl cellulose were added thereto, and mixed for 10 minutes. Then, magnesium stearate and talc were added thereto, and mixed for 5 minutes to prepare a powder. The powder was filled into a lower punch below a die using a feeder. Then, compression was performed using an upper punch, and a tablet was discharged from the die to prepare an ubidecarenone-containing monolayered tablet.

### (3) Preparation Example 1-3

### Formulation of Ubidecarenone-Containing Monolayered Tablet

TABLE 3

| Ingredient | Weight (per one tablet) | |
|---|---|---|
| Thiamine nitrate | 30.0 | mg |
| Biotin | 45.0 | μg |
| Riboflavin | 200.0 | mg |
| Ubidecarenone | 5.0 | mg |
| Low-substituted hydroxymethyl cellulose | 20.0 | mg |
| Ludipress | 200.0 | mg |
| Magnesium stearate | 10.7 | mg |
| Talc | 1.7 | mg |
| Silicone dioxide | 1.2 | mg |

Ubidecarenone were mixed with thiamine nitrate, biotin, riboflavin, low-substituted hydroxymethyl cellulose, and ludipress for 10 minutes, and then magnesium stearate, talc, and anhydrous silicic acid were added thereto, followed by mixing for 5 minutes. The mixed powder was filled into a lower punch below a die using a feeder. Then, compression was performed using an upper punch, and a tablet was discharged from the die to prepare an ubidecarenone-containing monolayered tablet

### (4) Preparation Example 1-4

### Formulation of Ubidecarenone-Containing Monolayered Tablet

TABLE 4

| Ingredient | Weight (per one tablet) | |
|---|---|---|
| Ubidecarenone | 5.0 | mg |
| Ascorbic acid | 100.0 | mg |
| Alpha-tocopherol acetate | 10.0 | I.U. |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone, alpha-tocopherol acetate, and lactose were granulated using povidone as a binder, and then sieved. Then, low-substituted hydroxymethyl cellulose and ascorbic acid were added thereto, and mixed for 10 minutes. Magnesium stearate, talc, and silicone dioxide were added thereto, and mixed for 5 minutes. The mixed powder was filled into a lower punch below a die using a feeder. Then, compression was performed using an upper punch, and a tablet was discharged from the die to prepare a ubidecarenone-containing monolayered tablet.

### (5) Preparation Example 1-5

### Formulation of Ubidecarenone-Containing Monolayered Tablet

TABLE 5

| Ingredient | Weight (per one tablet) | |
|---|---|---|
| Ubidecarenone | 5.0 | mg |
| Choline bitartrate | 50.0 | mg |
| Alpha-tocopherol acetate | 10.0 | I.U. |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone, choline bitartrate, alpha-tocopherol acetate, and lactose were granulated using povidone as a binder, and then sieved. Low-substituted hydroxymethyl cellulose was added thereto, and mixed for 10 minutes. Magnesium stearate, talc, and silicone dioxide were added thereto, and mixed for 5 minutes. The mixed powder was filled into a lower punch below a die using a feeder. Then, compression was performed using an upper punch, and a tablet was discharged from the die to prepare an ubidecarenone-containing monolayered tablet.

### (6) Preparation Example 1-6

### Formulation of Ubidecarenone-Containing Monolayered Tablet

TABLE 6

| Ingredient | Weight (per one tablet) | |
|---|---|---|
| Ubidecarenone | 5.0 | mg |
| Choline bitartrate | 50.0 | mg |
| Retinol palmitate | 500.0 | I.U. |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone, choline bitartrate, and lactose were granulated using povidone as a binder, and then sieved. Retinol palmitate and low-substituted hydroxymethyl cellulose were added thereto, and mixed for 10 minutes. Magnesium stearate, talc, and silicone dioxide were added thereto, and mixed fro 5 minutes. The mixed powder was filled into a lower punch below a die using a feeder. Then, compression was performed using an upper punch, and a tablet was discharged from the die to prepare a ubidecarenone-containing monolayered tablet.

### (7) Preparation Example 1-7

### Formulation of Ubidecarenone-Containing Monolayered Tablet

TABLE 7

| Ingredient | Weight (per one tablet) | |
|---|---|---|
| Ubidecarenone | 5.0 | mg |
| 0.1% Cyanocobalamine | 5.0 | mg |
| Choline bitartrate | 50.0 | mg |
| Low-substituted hydroxymethyl Cellulose | 20.0 | mg |
| Ludipress | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone was mixed with 0.1% cyanocobalamine, choline bitartrate, low-substituted hydroxymethyl cellulose, ludipress, and povidone for 10 minutes, and magnesium stearate, talc, and silicone dioxide were added thereto, followed by mixing for 5 minutes. The mixed powder was filled into a lower punch below a die using a feeder. Then, compression was performed using an upper punch, and a tablet was discharged from the die to prepare a ubidecarenone-containing monolayered tablet.

### 2. Test Example 1

### Stability Test for Each Formulation of Preparation Examples 1-1 to 1-7

(1) Result of Accelerated Stability Test for Ubidecarenone in Monolayered Tablet (Unit: % Residual Amount of Ubidecarenone)

TABLE 8

| Test Sample | Initiation day | 2 months | 4 months | 6 months |
|---|---|---|---|---|
| Preparation | 100.0 | 98.3 | 95.6 | 92.1 |
| Example 1-1 | | | | |
| Preparation | 100.0 | 97.8 | 94.5 | 91.7 |
| Example 1-2 | | | | |
| Preparation | 100.0 | 96.5 | 93.7 | 90.2 |
| Example 1-3 | | | | |
| Preparation | 100.0 | 77.7 | 58.1 | 39.4 |
| Example 1-4 | | | | |
| Preparation | 100.0 | 78.9 | 64.4 | 46.3 |
| Example 1-5 | | | | |
| Preparation | 100.0 | 86.3 | 74.8 | 68.6 |
| Example 1-6 | | | | |
| Preparation | 100.0 | 76.2 | 61.4 | 40.1 |
| Example 1-7 | | | | |

(2) Result of Long-Term Stability Test for Ubidecarenone in Monolayered Tablet (Unit: % Residual Amount of Ubidecarenone)

TABLE 9

| Test | Initiation | 3 | 6 | 12 | 18 | 24 |
|---|---|---|---|---|---|---|
| Sample | day | months | months | months | months | months |
| Preparation | 100.0 | 99.7 | 99.4 | 98.6 | 97.3 | 96.8 |
| Example 1-1 | | | | | | |
| Preparation | 100.0 | 98.8 | 98.0 | 96.8 | 95.8 | 94.2 |
| Example 1-2 | | | | | | |
| Preparation | 100.0 | 99.5 | 97.7 | 97.2 | 95.2 | 94.8 |
| Example 1-3 | | | | | | |
| Preparation | 100.0 | 82.7 | 67.6 | 49.7 | 32.4 | 15.2 |
| Example 1-4 | | | | | | |
| Preparation | 100.0 | 88.9 | 73.1 | 54.5 | 48.9 | 21.8 |
| Example 1-5 | | | | | | |
| Preparation | 100.0 | 95.3 | 90.8 | 86.0 | 74.6 | 59.8 |
| Example 1-6 | | | | | | |
| Preparation | 100.0 | 89.2 | 76.7 | 57.8 | 37.4 | 22.6 |
| Example 1-7 | | | | | | |

As shown in [Table 8] and [Table 9], the tablets prepared by the formulations of Preparation Examples 1-1, 1-2, and 1-3 were subjected to the accelerated stability test for 6 months or long-term stability test for 24 months. As a result, high stability of 90% or more was maintained, and thus they can be used as a composition of the ubidecarenone-containing layer of the present invention.

The tablets prepared by the formulations of Preparation Examples 1-4, 1-5, 1-6, and 1-7 were also subjected to the accelerated stability test for 6 months or long-term stability test for 24 months. However, the content of ubidecarenone was excessively reduced, and thus they cannot be used as a composition of the ubidecarenone-containing layer of the present invention. Accordingly, these results suggest that some or all of other active ingredients than ubidecarenone should be contained in the other layer separated from the ubidecarenone-containing layer in the multi-layered tablets of the present invention.

II. Preparation of Multi-Layered Tablet of the Present Invention and Monolayered Tablet of Prior Art and their Stability Test

1. Preparation of Multi-Layered Tablet of the Present Invention

### (1) Preparation Example 2

### Preparation of Bilayered Tablet A Consisting of Ubidecarenone-Containing Layer/Vitamin and Mineral-Containing Layer

(A) Ubidecarenone-Containing Layer

TABLE 10

| Ingredient | Weight (per one tablet) | |
|---|---|---|
| Low-substituted hydroxymethyl Cellulose | 20.0 | mg |
| Ludipress | 200.0 | mg |
| Thiamine nitrate | 30.0 | mg |
| Biotin | 45.0 | μg |
| Ubidecarenone | 5.0 | mg |
| Magnesium stearate | 10.7 | mg |
| Talc | 1.7 | mg |
| Silicone dioxide | 1.2 | mg |

(B) Vitamin and Mineral-Containing Layer

TABLE 11

| Ingredient | Weight (per one tablet) | |
|---|---|---|
| Retinol palmitate | 500.0 | I.U. |
| Riboflavin | 12.0 | mg |
| Nicotinamide | 10.0 | mg |
| Calcium pantothenate | 30.0 | mg |
| Pyridoxine hydrochloride | 10.0 | mg |
| Dried cholecalciferol | 1.0 | mg |
| 0.1% Cyanocobalamine | 5.0 | mg |
| Alpha-tocopherol acetate | 10.0 | I.U. |
| Choline bitartrate | 25.0 | mg |
| Calcium hydrogen phosphate | 50.0 | mg |
| Low-substituted hydroxymethyl cellulose | 20.9 | mg |
| Lactose | 110.2 | mg |
| Ludipress | 173.4 | mg |
| Povidone | 2.5 | mg |
| Magnesium stearate | 25.5 | mg |
| Talc | 4.1 | mg |
| Silicone dioxide | 2.9 | mg |

(C) Preparation Method

Ubidecarenone was mixed with thiamine nitrate, biotin, low-substituted hydroxymethyl cellulose, and ludipress for 10 minutes, and magnesium stearate, talc, and silicone dioxide were added thereto, followed by mixing for 5 minutes to prepare a mixture of the ubidecarenone-containing layer (A). Choline bitartrate, calcium hydrogen phosphate, and lactose were granulated using povidone as a binder, and sieved. Riboflavin, nicotinamide, calcium pantothenate, pyridoxine hydrochloride, dried cholecalciferol, 0.1% cyanocobalamine, alpha-tocopherol acetate, ludipress, low-substituted hydroxymethyl cellulose, and povidone were added thereto, and mixed for 10 minutes. Then, magnesium stearate, talc, and silicone dioxide were added thereto, and mixed to prepare a mixture of the vitamin and mineral-containing layer (B).

The mixture of the ubidecarenone-containing layer (A) was filled into a lower punch below a die, and a predetermined pressure was applied thereto using an upper punch for a primary tableting. Subsequently, the mixed powder of vitamin and mineral-containing layer (B) was filled, and subjected to final compression. A tablet was discharged from the die to prepare an oral bilayered vitamin complex tablet containing ubidecarenone.

### (2) Preparation Example 3

### Preparation of Bilayered Tablet B Consisting of Ubidecarenone-Containing Layer/Vitamin and Mineral-Containing Layer

(A) Ubidecarenone-Containing Layer

TABLE 12

| Ingredient | Weight (per one tablet) | |
|---|---|---|
| Low-substituted hydroxymethyl Cellulose | 20.0 | mg |
| Ludipress | 200.0 | mg |
| Ubidecarenone | 5.0 | mg |
| Magnesium stearate | 10.7 | mg |
| Talc | 1.7 | mg |
| Silicone dioxide | 1.2 | mg |

(B) Vitamin and Mineral-Containing Layer

TABLE 13

| Ingredient | Weight (per one tablet) | |
|---|---|---|
| Thiamine nitrate | 30.0 | mg |
| Retinol palmitate | 500.0 | I.U. |
| Biotin | 45.0 | ug |
| Riboflavin | 12.0 | mg |
| Nicotinamide | 10.0 | mg |
| Calcium pantothenate | 30.0 | mg |
| Pyridoxine hydrochloride | 10.0 | mg |
| Dried Cholecalciferol | 1.0 | mg |
| 0.1% Cyanocobalamine | 5.0 | mg |
| Alpha-tocopherol acetate | 10.0 | I.U. |
| Choline bitartrate | 25.0 | mg |
| Calcium hydrogen phosphate | 50.0 | mg |
| Low-substituted hydroxymethyl cellulose | 20.9 | mg |
| Lactose | 110.2 | mg |
| Ludipress | 173.4 | mg |
| Povidone | 2.5 | mg |
| Magnesium stearate | 25.5 | mg |
| Talc | 4.1 | mg |
| Silicone dioxide | 2.9 | mg |

(C) Preparation Method

Ubidecarenone was mixed with low-substituted hydroxymethyl cellulose, and ludipress for 10 minutes, and magnesium stearate, talc, and silicone dioxide were added thereto, followed by mixing for 5 minutes to prepare a mixture of the ubidecarenone-containing layer (A). Biotin, choline bitartrate, calcium hydrogen phosphate and lactose were granulated using povidone as a binder, and sieved. Calcium pantothenate, retinol palmitate, thiamine nitrate, riboflavin, nicotinamide, pyridoxine hydrochloride, dried cholecalciferol, 0.1% cyanocobalamine, alpha-tocopherol acetate, low-substituted hydroxymethyl cellulose, and ludipress were added thereto, and mixed for 10 minutes. Then, magnesium stearate, talc, and silicone dioxide were added thereto, and mixed for 5 minutes to prepare a mixture of the vitamin and mineral-containing layer (B).

The mixture of the ubidecarenone-containing layer (A) was filled into a lower punch below a die, and a predetermined pressure was applied thereto using an upper punch for a primary tableting. Subsequently, the mixed powder of vitamin and mineral-containing layer (B) was filled, and subjected to final compression. A tablet was discharged from the die to prepare an oral bilayered vitamin complex tablet containing ubidecarenone.

### (3) Preparation Example 4

### Preparation of Core Tablet A Consisting of Ubidecarenone-Containing Inner Layer/Vitamin and Mineral-Containing Outer Layer

(A) Ingredients and Weight of Ubidecarenone-Containing Inner Layer

TABLE 14

| Ingredient | Weight (per one tablet) |
|---|---|
| Low-substituted hydroxymethyl cellulose | 20.0 mg |
| Lactose | 200.0 mg |
| Calcium pantothenate | 30.0 mg |
| Selenium (dried yeast) | 18.0 mg |
| Ubidecarenone | 5.0 mg |
| Povidone | 2.5 mg |
| Magnesium stearate | 10.7 mg |
| Talc | 1.7 mg |

(B) Vitamin and Mineral-Containing Outer Layer

TABLE 15

| Ingredient | Weight (per one tablet) | |
|---|---|---|
| Fursultiamine | 50.0 | mg |
| Riboflavin | 12.0 | mg |
| Nicotinamide | 10.0 | mg |
| Pyridoxine hydrochloride | 10.0 | mg |
| Dried cholecalciferol | 1.0 | mg |
| 0.1% Cyanocobalamine | 5.0 | mg |
| Alpha-tocopherol acetate | 10.0 | I.U. |
| Retinol palmitate | 500.0 | I.U. |
| Gamma-oryzanol | 5.0 | mg |
| Ascorbic acid | 500.0 | mg |
| Inositol | 30.0 | mg |
| Low-substituted hydroxymethyl cellulose | 20.9 | mg |
| Lactose | 110.2 | mg |
| Microcrystalline cellulose | 173.4 | mg |
| Povidone | 5.0 | mg |
| Magnesium stearate | 25.5 | mg |
| Talc | 4.1 | mg |
| Silicone dioxide | 2.9 | mg |

(C) Preparation Method

Ubidecarenone was mixed with selenium, calcium pantothenate, lactose, low-substituted hydroxymethyl cellulose for 10 minutes, and povidone, magnesium stearate, and talc were added thereto, followed by mixing for 5 minutes to prepare a mixture of the ubidecarenone-containing inner layer (A).

Separately, fursultiamine, riboflavin, nicotinamide, pyridoxine hydrochloride, dried cholecalciferol, 0.1% cyanocobalamine, alpha-tocopherol acetate, retinol palmitate, ν(gamma)-oryzanol, ascorbic acid, inositol, lactose, low-substituted hydroxymethyl cellulose, microcrystalline cellulose, and povidone were added, and mixed for 10 minutes. Magnesium stearate, talc, and silicone dioxide were added thereto, and mixed to prepare a mixture of vitamin and mineral-containing outer layer (B).

The mixture of the ubidecarenone-containing inner layer (A) was filled into a lower punch below a die, and a predetermined pressure was applied thereto using an upper punch for tableting. Subsequently, the mixture of vitamin and mineral-containing outer layer (B) was mixed and filled into a lower punch, and subjected to final compression using an upper punch. A tablet was discharged from the die to prepare an oral vitamin complex core tablet containing ubidecarenone.

### (4) Preparation Example 5

### Preparation of Core Tablet B Consisting of Ubidecarenone-Containing Inner Layer/Vitamin and Mineral-Containing Outer Layer

(A) Ubidecarenone-Containing Inner Layer

TABLE 16

| Ingredient | Weight (per one tablet) |
|---|---|
| Low-substituted hydroxymethyl cellulose | 20.0 mg |
| Lactose | 200.0 mg |
| Ubidecarenone | 5.0 mg |
| Povidone | 2.5 mg |
| Magnesium stearate | 10.7 mg |
| Talc | 1.7 mg |

(B) Vitamin and Mineral-Containing Outer Layer

TABLE 17

| | Weight |
|---|---|
| Ingredient | (per one tablet) |
| Ascorbic acid | 200.0 mg |
| L-cysteine | 120.0 mg |
| Calcium pantothenate | 12.0 mg |
| Microcrystalline cellulose | 50.0 mg |
| Ludipress | 93.4 mg |
| Low-substituted hydroxymethyl cellulose | 20.9 mg |
| Magnesium stearate | 25.5 mg |
| Talc | 4.1 mg |
| Silicone dioxide | 2.9 mg |

(C) Preparation Method

Ubidecarenone was mixed with lactose and low-substituted hydroxymethyl cellulose for 10 minutes, and then povidone, magnesium stearate, and talc were added thereto, followed by mixing for 5 minutes to prepare a mixture of ubidecarenone-containing inner layer (A).

Separately, ascorbic acid, L-cysteine, calcium pantothenate, microcrystalline cellulose, ludipress, and low-substituted hydroxymethyl cellulose were added, and mixed for 10 minutes. Magnesium stearate, talc, and silicone dioxide were added thereto, and mixed for 5 minutes to prepare a mixture of vitamin and mineral-containing outer layer (B).

The mixture of the ubidecarenone-containing inner layer (A) was filled into a lower punch below a die, and a predetermined pressure was applied thereto using an upper punch for tableting. Subsequently, the mixture of vitamin and mineral-containing outer layer (B) was mixed and filled into a lower punch, and subjected to final compression using an upper punch. A tablet was discharged from the die to prepare an oral vitamin complex core tablet containing ubidecarenone.

### (5) Preparation Example 6

### Preparation of Three-Layered Tablet Consisting of Ubidecarenone Layer/Vitamin Layer/Mineral Layer

(A) Ubidecarenone-Containing Layer

TABLE 18

| | Weight |
|---|---|
| Ingredient | (per one tablet) |
| Low-substituted hydroxymethyl Cellulose | 20.0 mg |
| Lactose | 200.0 mg |
| Ubidecarenone | 5.0 mg |
| Povidone | 2.5 mg |
| Magnesium stearate | 10.7 mg |
| Talc | 1.7 mg |
| Silicone dioxide | 3.2 mg |

(B) Layer Containing Vitamin Except Ubidecarenone

TABLE 19

| Ingredient | Weight (per one tablet) | |
|---|---|---|
| Riboflavin | 12.0 | mg |
| Nicotinamide | 10.0 | mg |
| Calcium pantothenate | 30.0 | mg |
| Pyridoxine hydrochloride | 10.0 | mg |
| Dried cholecalciferol | 1.0 | mg |
| 0.1% Cyanocobalamine | 5.0 | mg |
| Alpha-tocopherol acetate | 10.0 | I.U. |
| Retinol palmitate | 500.0 | I.U. |
| Ascorbic acid | 500.0 | mg |
| Inositol | 30.0 | mg |
| Low-substituted hydroxymethyl cellulose | 20.9 | mg |
| Lactose | 110.2 | mg |
| Microcrystalline cellulose | 173.4 | mg |
| Povidone | 5.0 | mg |
| Magnesium stearate | 25.5 | mg |
| Talc | 4.1 | mg |
| Silicone dioxide | 2.9 | mg |

(C) Layer Containing Mineral Except Ubidecarenone

TABLE 20

| | Weight |
|---|---|
| Ingredient | (per one tablet) |
| Choline bitartrate | 25.0 mg |
| Calcium hydrogen phosphate | 10.0 mg |
| Zinc sulfate | 50.0 mg |
| Dried iron sulfate | 12.0 mg |
| Selenium (dried yeast) | 18.0 mg |
| Low-substituted hydroxymethyl cellulose | 20.9 mg |
| Lactose | 110.2 mg |
| Microcrystalline cellulose | 173.4 mg |
| Povidone | 5.0 mg |
| Magnesium stearate | 25.5 mg |
| Talc | 4.1 mg |
| Silicone dioxide | 2.9 mg |

(D) Preparation Method

Ubidecarenone and lactose were granulated using povidone as a binder, and sieved. Low-substituted hydroxymethyl cellulose was added thereto, and mixed for 10 minutes. Magnesium stearate, talc, and silicone dioxide were added thereto, and mixed for 5 minutes to prepare a mixture of ubidecarenone-containing layer (A).

Riboflavin, nicotinamide, calcium pantothenate, pyridoxine hydrochloride, dried cholecalciferol, 0.1% cyanocobalamine, alpha-tocopherol acetate, retinol palmitate, ascorbic acid, inositol, low-substituted hydroxymethyl cellulose, lactose, micro-crystalline cellulose, povidone were mixed for 10 minutes, and magnesium stearate, talc, and silicone dioxide were added thereto, followed by mixing for 5 minutes to prepare a mixture of layer containing vitamin except ubidecarenone (B).

Separately, choline bitartrate, calcium hydrogen phosphate, zinc sulfate, dried iron sulfate, selenium, lactose were granulated using povidone as a binder, and sieved. Microcrystalline cellulose and low-substituted hydroxymethyl cellulose were mixed, and then magnesium stearate, talc, and silicone dioxide were added thereto, followed by mixing for 5 minutes to prepare a mixture of mineral-containing layer (C).

The mixture of the ubidecarenone-containing inner layer (A) was filled into a lower punch below a die, and a predetermined pressure was applied thereto using an upper punch for a primary tableting. Subsequently, the mixed powder of vitamin-containing layer (B) was filled, and subjected to compression. Then, the mixed powder of mineral-containing layer (C) was filled, and subjected to compression, and a tablet was discharged from the die to prepare an oral three-layered vitamin complex tablet containing ubidecarenone.

### (6) Preparation Example 7

### Preparation of Film Coated Bilayered Tablet Containing Ubidecarenone

A bilayered tablet was prepared according to the formulation of Preparation Example 2, and then 44.0 g of polyvinyl alcohol, 20.0 g of talc, 15.7 g of allura red AC aluminum lake, 12.4 g of polyethylene glycol, 3.5 g of lecithin, 3.0 g of titanium oxide, 1.2 g of erythrosine, and 0.3 g of indigo carmine aluminum lake were dissolved in purified water, and film-coated to prepare a coated tablet with 32 mg coating (per tablet).

2. Preparation of Multi-Layered Tablet by Prior Art

### (1) Comparative Preparation Example 1

### Preparation of Monolayered Tablet Containing Ubidecarenone, Vitamin and Mineral (Comparison with Bilayered Tablet A of Preparation Example 2)

The mixture of ubidecarenone-containing layer (A) of Table 10 prepared in Preparation Example 2 and the mixture of layer containing vitamin and mineral except ubidecarenone (B) of Table 11 were mixed with each other, and then filled into the lower punch below the die using the feeder, and then subjected to compression using the upper punch. A tablet was discharged from the die to prepare a monolayered tablet containing ubidecarenone.

### (2) Comparative Preparation Example 2

### Preparation of Monolayered Tablet Containing Ubidecarenone, Vitamin and Mineral (Comparison with Core Tablet A of Preparation Example 4)

The mixture of ubidecarenone-containing layer (A) of Table 14 prepared in Preparation Example 4 and the mixture of layer containing vitamin and mineral except ubidecarenone (B) of Table 15 were mixed with each other, and then filled into the lower punch below the die using the feeder, and then subjected to compression using the upper punch. A tablet was discharged from the die to prepare a monolayered tablet containing ubidecarenone.

### (3) Comparative Preparation Example 3

### Preparation of Monolayered Tablet Containing Ubidecarenone, Vitamin and Mineral (Comparison with Core Tablet B of Preparation Example 5)

The mixture of ubidecarenone-containing layer (A) of Table 16 prepared in Preparation Example 5 and the mixture of layer containing vitamin and mineral except ubidecarenone (B) of Table 17 were mixed with each other, and then filled into the lower punch below the die using the feeder, and then subjected to compression using the upper punch. A tablet was discharged from the die to prepare a monolayered tablet containing ubidecarenone.

### (4) Comparative Preparation Example 4

### Preparation of Monolayered Tablet Containing Ubidecarenone, Vitamin and Mineral (Comparison with Three-Layered Tablet of Preparation Example 6)

The mixture of ubidecarenone-containing layer (A) of Table 18 prepared in Preparation Example 6, the mixture of layer containing vitamin except ubidecarenone (B) of Table 19, and the mixture of layer containing mineral (C) of Table 20 were mixed with each other, and then filled into the lower punch below the die using the feeder, and then subjected to compression using the upper punch. A tablet was discharged from the die to prepare a monolayered tablet containing ubidecarenone.

3. Preparation of Monolayered Tablet by Separating Ubidecarenone from Vitamins According to Granulation Method in Prior Art

### (A) Comparative Preparation Example 5-1

### Monolayered Tablet I Prepared by Separating Ubidecarenone from Vitamins According to Granulation Method

TABLE 21

| Ingredient | Weight (per one tablet) | |
|---|---|---|
| Ubidecarenone | 5.0 | mg |
| Ascorbic acid | 100.0 | mg |
| Alpha-tocopherol acetate | 10.0 | I.U. |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone and lactose were granulated using povidone as a binder, and sieved. Low-substituted hydroxymethyl cellulose, ascorbic acid, and alpha-tocopherol acetate were added thereto, and mixed for 10 minutes. Magnesium stearate, talc, and silicone dioxide were added thereto, and mixed for 5 minutes to prepare a mixture containing ubidecarenone.

The mixed powder was filled into the lower punch below the die using a feeder, and then subjected to compression using the upper punch. A tablet was discharged from the die to prepare a monolayered tablet containing ubidecarenone.

### (B) Comparative Preparation Example 5-2

### Monolayered Tablet II Prepared by Separating Ubidecarenone from Vitamins According to Granulation Method

TABLE 22

| Ingredient | Weight (per one tablet) | |
|---|---|---|
| Ubidecarenone | 5.0 | mg |
| Ascorbic acid | 100.0 | mg |
| Alpha-tocopherol acetate | 10.0 | I.U |
| Choline bitartrate | 50.0 | mg |
| Low-substituted hydroxymethyl cellulose | 20.0 | mg |
| Lactose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone and low-substituted hydroxymethyl cellulose were granulated using povidone as a binder, and sieved. Lactose, ascorbic acid, alpha-tocopherol acetate, and choline bitartrate were added thereto, and mixed for 10 minutes. Magnesium stearate, talc, and silicone dioxide were added thereto, and mixed for 5 minutes to prepare a mixture containing ubidecarenone.

The mixture was filled into the lower punch below the die using a feeder, and then subjected to compression using the upper punch. A tablet was discharged from the die to prepare a monolayered tablet containing ubidecarenone.

### (C) Comparative Preparation Example 5-3

### Monolayered Tablet III Prepared by Separating Ubidecarenone from Vitamins According to Granulation Method

TABLE 23

| Ingredient | Weight (per one tablet) | |
|---|---|---|
| Ubidecarenone | 5.0 | mg |
| Choline bitartrate | 50.0 | mg |
| Alpha-tocopherol acetate | 10.0 | I.U. |
| Ascorbic acid | 100.0 | mg |
| Low-substituted hydroxymethyl cellulose | 20.0 | mg |
| Microcrystalline cellulose | 200.0 | mg |
| Povidone | 2.5 | mg |
| Magnesium stearate | 10.7 | mg |
| Talc | 1.7 | mg |
| Silicone dioxide | 3.2 | mg |

Ubidecarenone and microcrystalline cellulose were granulated using povidone as a binder, and sieved. Low-substituted hydroxymethyl cellulose, ascorbic acid, alpha-tocopherol acetate, and choline bitartrate were added thereto, and mixed for 10 minutes. Magnesium stearate, talc, and silicone dioxide were added thereto, and mixed for 5 minutes to prepare a mixture containing ubidecarenone.

The mixture was filled into the lower punch below the die using a feeder, and then subjected to compression using the upper punch. A tablet was discharged from the die to prepare a monolayered tablet containing ubidecarenone.

### 4. Test Example 2

### Accelerated Stability Test of Preparation Examples 2 to 6 (Present Invention) and Comparative Preparation Examples 1 to 4 (Prior Art)

The tablets prepared in Preparation Examples 2, 3, 4, 5, and 6 were subjected to the accelerated stability test with the tablets prepared in Comparative Preparation Examples 1, 2, 3, and 4 as control groups. The prepared tablets was packaged into a HDPE (High density polyethylene) bottle, and stored under the conditions of 40±2° C., 75±5% RH, respectively. Then, changes in the content were measured at initiation day, 2, 4, and 6 month. The result was represented by a relative residual amount, when the content of ubidecarenone on the initiation day of accelerated stability test was regarded as 100.0%.

TABLE 24
[Table 24: Result of accelerated stability test for tablets containing
ubidecarenone]

| | Initiation | | | |
|---|---|---|---|---|
| Test Sample | day | 2 months | 4 months | 6 months |
| Preparation Example 2 | 100.0 | 97.0 | 94.2 | 90.7 |
| (bilayered tablet A) | | | | |
| Preparation Example 3 | 100.0 | 98.6 | 96.1 | 93.9 |
| (bilayered tablet B) | | | | |
| Preparation Example 4 | 100.0 | 97.3 | 93.5 | 91.4 |
| (core tablet A) | | | | |
| Preparation Example 5 | 100.0 | 96.5 | 91.7 | 91.1 |
| (core tablet B) | | | | |
| Preparation Example 6 | 100.0 | 97.7 | 93.8 | 92.0 |
| (three-layered tablet) | | | | |
| Comparative Preparation | 100.0 | 68.9 | 32.4 | 15.2 |
| Example 1 (monolayered | | | | |
| tablet) | | | | |
| Comparative Preparation | 100.0 | 74.3 | 51.8 | 33.6 |
| Example 2 (monolayered | | | | |
| tablet) | | | | |
| Comparative Preparation | 100.0 | 77.5 | 60.9 | 43.1 |
| Example 3 (monolayered | | | | |
| tablet) | | | | |
| Comparative Preparation | 100.0 | 67.0 | 40.1 | 22.7 |
| Example 4 (monolayered | | | | |
| tablet) | | | | |

(Unit: % residual amount of ubidecarenone)

When the total ingredients of Preparation Examples 2 to 6 were compared to the monolayered tablets in Comparative Preparation Examples 1, 2, 3, and 4, the stability of ubidecarenone was found to be improved in Preparation Examples 2, 3, 4, 5, and 6, where the ubidecarenone layer was separated from the vitamin/mineral layer.

### 5. Test Example 3

### Accelerated Stability Test of Monolayered Tablets (Prior Art) Separated by Granulation Method

TABLE 25
[Table 25: Result of accelerated stability test for compositions of
ubidecarenone layer]

| Test Sample | Initiation day | 2 months | 4 months | 6 months |
|---|---|---|---|---|
| Preparation Example | 100.0 | 77.7 | 58.1 | 39.4 |
| 1-4 | | | | |
| Comparative | 100.0 | 86.2 | 74.5 | 58.3 |
| Preparation Example | | | | |
| 5-1 | | | | |
| Comparative | 100.0 | 82.1 | 70.4 | 61.3 |
| Preparation Example | | | | |
| 5-2 | | | | |
| Comparative | 100.0 | 84.8 | 71.1 | 52.7 |
| Preparation Example | | | | |
| 5-3 | | | | |
| Preparation Example 3 | 100.0 | 98.6 | 96.1 | 93.9 |

(Unit: % residual amount of ubidecarenone)

Tablets prepared in Comparative Preparation Examples 5-1 to 5-3 were stabilized by granulating ubidecarenone with excipients. They are more stable than that prepared in Preparation Example 1-4, but the content of ubidecarenone was found to be excessively reduced in 6 month accelerated stability test, and less stable than the bilayered tablet prepared in Preparation Example 3. Thus, these results suggest that the stabilization of ubidecarenone in the complex vitamin tablet cannot be achieved by the simple granulation method.

III. Result of Stability Test and Relationship with Contents of Ubidecarenone and Vitamins Under Co-Existence

In accordance with the regulations set forth by the Korea Pharmacopeia, preparation of medicines such vitamins and minerals should meet the preparation standard. In particular, the efficacy and effect of the ingredient can be labeled on the product, when the content of the ingredients in the product is more than a predetermined amount. Thus, the present inventors examined the ingredients decreasing the stability of ubidecarenone, which are regarded as reference. That is, even though the ingredients rarely inhibit the stability of ubidecarenone when used in a small amount, the ingredients that inhibit the stability of ubidecarenone when used in an amount over the standard were regarded as ingredients decreasing the stability of ubidecarenone.

The detailed description is as follows.

1. Stability Test for Ubidecarenone and Vitamins/Minerals Under Co-Existence

First, ubidecarenone was mixed with vitamins or minerals that are typically used for formulation at a ratio of 1:1, and the changes in the content of ubidecarenone were measured under the conditions of 40±2° C., 75±5 RH % for 6 weeks. The result was expressed as a relative residual amount, when the content of ubidecarenone on the initiation day of accelerated stability test was regarded as 100.0%, as shown in the following Table 26.

TABLE 26
[Table 26: Result of stability test for ubidecarenone and vitamins
under co-existence]

| | | 2 | 4 | 6 |
|---|---|---|---|---|
| Mixture | Initial | weeks | weeks | weeks |
| Ubidecarenone | 100 | 99.2 | 97.2 | 96.4 |
| Ubidecarenone + Retinol palmitate | 100 | 97.7 | 90.7 | 76.2 |
| Ubidecarenone + Choline bitartrate | 100 | 84.4 | 73.0 | 58.0 |
| Ubidecarenone + Biotin | 100 | 98.3 | 94.2 | 90.2 |
| Ubidecarenone + Ascorbic acid | 100 | 94.7 | 92.2 | 87.2 |
| Ubidecarenone + 0.1% | 100 | 83.5 | 76.6 | 74.4 |
| Cyanocobalamine | | | | |
| Ubidecarenone + Dried | 100 | 90.8 | 89.9 | 75.5 |
| cholecalciferol | | | | |
| Ubidecarenone + Thiamine nitrate | 100 | 98.6 | 94.7 | 90.4 |
| Ubidecarenone + Fursultiamine | 100 | 84.5 | 79.4 | 75.4 |
| Ubidecarenone + Pyridoxine | 100 | 91.1 | 84.9 | 74.6 |
| hydrochloride | | | | |
| Ubidecarenone + Nicotinamide | 100 | 92.5 | 86.2 | 80.2 |
| Ubidecarenone + Calcium | 100 | 98.3 | 96.0 | 92.3 |
| pantothenate | | | | |
| Ubidecarenone + Riboflavin | 100 | 97.7 | 95.3 | 90.6 |
| Ubidecarenone + Alpha-tocopherol | 100 | 96.4 | 93.9 | 72.3 |
| acetate | | | | |
| Ubidecarenone + Inositol | 100 | 90.5 | 80.1 | 72.6 |

(Unit: % residual amount of ubidecarenone)

As shown in Table 26, when each ingredient was singly mixed with ubidecarenone, choline bitartrate, alpha-tocopherol acetate, dried cholecalciferol, and retinol palmitate were found to excessively reduce the stability of ubidecarenone.

2. Stability Test for Ubidecarenone and Vitamins/Minerals Under Co-Existence According to Typical Formulation Ratio

Ubidecarenone and vitamins/minerals were under co-existence at a typical formulation amount and ratio, and then the accelerated stability test for ubidecarenone was performed. The results are as follows. In the following table, ubidecarenone is contained at the same amount of 50 mg in each formulation. The amount of ubidecarenone was omitted in the column of preparation formulation.

TABLE 27

| | Preparation | | | | |
|---|---|---|---|---|---|
| | formulation | Initial | 2 weeks | 4 weeks | 6 weeks |
| Mixture | (mg) | (%) | (%) | (%) | (%) |
| Ubidecarenone alone | 50 | 100 | 99.2 | 97.2 | 96.4 |
| Thiamine nitrate- | 30050 | 100 | 97.2 | 97.0 | 96.8 |
| Ubidecarenone | | | | | |
| Fursultiamine | 30050 | 100 | 90.4 | 83.4 | 74.3 |
| Ubidecarenone | | | | | |
| RiboflavinUbidecarenone | 12050 | 100 | 98.5 | 98.0 | 96.4 |
| NicotinamideUbidecarenone | 10050 | 100 | 85.2 | 79.1 | 71.4 |
| Calcium pantothenate- | 12050 | 100 | 97.9 | 96.6 | 94.4 |
| Ubidecarenone | | | | | |
| Pyridoxine hydrochloride- | 10050 | 100 | 91.8 | 85.8 | 73.2 |
| Ubidecarenone | | | | | |
| Dried cholecalciferol- | 1050 | 100 | 92.1 | 84.4 | 76.4 |
| Ubidecarenone | | | | | |
| 0.1% Cyanocobalamine- | 5050 | 100 | 83.5 | 76.6 | 74.4 |
| Ubidecarenone | | | | | |
| 90% Ascorbic acid- | 500050 | 100 | 89.4 | 73.6 | 54.2 |
| Ubidecarenone | | | | | |
| Alpha-tocopherol | 20050 | 100 | 90.7 | 74.2 | 68.3 |
| acetateUbidecarenone | | | | | |
| Retinol palmitate- | 1050 | 100 | 93.4 | 87.3 | 76.4 |
| Ubidecarenone | | | | | |
| BiotinUbidecarenone | 4550 | 100 | 98.9 | 96.2 | 94.7 |
| InositolUbidecarenone | 100050 | 100 | 85.2 | 73.8 | 61.4 |

The content of ingredients such as vitamins and the content of ubidecarenone (50 mg equally) under the co-existence were represented in the column of preparation formulation of Table 27.

As such, when the amount contained in the product was regarded as content, the relative residual amount of ubidecarenone was found to be reduced by 90% or less in the accelerated stability test for some ingredients. Accordingly, it can be seen that the multi-layered tablet of the present invention should be prepared by separating the ingredients from the ubidecarenone-containing layer.

3. Stability Test for Ubidecarenone According to Change in the Content (%) of Vitamin C Having a High Daily Dosage

10 mg of ubidecarenone and 50 mg, 100 mg, 500 mg, 1000 mg, and 1500 mg of 90% ascorbic acid were contained under the co-existence to prepare each sample, and the accelerated stability test was performed. The results are as follows.

TABLE 28

| Content of 90% | Initial (% | 2 weeks (% | 4 weeks (% | 6 weeks (% |
|---|---|---|---|---|
| ascorbic acid in | residual | residual | residual | residual |
| sample (mg) | amount) | amount) | amount) | amount) |
| 0 | 100 | 99.2 | 97.2 | 96.4 |
| 50 | 100 | 96.4 | 92.5 | 84.3 |
| 100 | 100 | 89.4 | 73.6 | 54.2 |
| 500 | 100 | 73.7 | 48.1 | 42.2 |
| 1000 | 100 | 78.9 | 34.4 | 36.3 |
| 1500 | 100 | 71.3 | 58.8 | 28.6 |

As such, it was found that as the content of ascorbic acid in the sample tablet increases, the residual amount of ubidecarenone was remarkably reduced. Considering that the typical vitamin formulation contains vitamin C in an amount of 500 to 5000 mg, vitamin C should be regarded as the ingredient decreasing the stability of ubidecarenone, demonstrating that the multi-layered tablet of the present invention should be prepared by separating ubidecarenone therefrom.

### INDUSTRIAL APPLICABILITY

The present invention provides a vitamin/mineral complex tablet comprising ubidecarenone useful for the human body, in particular, an oral solid formulation having enhanced stability and bioavailability of ubidecarenone. In the present invention, the multi-layered tablet is prepared by a simple process without addition of stabilizer such as organic acids, thereby increasing the stability of ubidecarenone.

The effect can be achieved in such a manner that a first layer contains ubidecarenone and other pharmaceutical composition not decreasing the stability of ubidecarenone, and other layer contains vitamins and minerals decreasing the stability of ubidecarenone, thereby minimizing the contact of each ingredient. In addition, moisture-proof coating is applied to the tablet, and thus provides the effect of preventing additional degradation and oxidation of ubidecarenone due to moisture.

1. An ubidecarenone-containing multi-layered vitamin complex tablet, comprising:
a first layer containing ubidecarenone as a first ingredient; and
a second layer containing an active ingredient as a second ingredient, said active ingredient decreasing the stability of ubidecarenone;
wherein said second ingredient contains vitamin selected from the group consisting of ascorbic acid, alpha-tocopherol acetate, retinol palmitate, cyanocobalamine, nicotinamide, pyridoxine hydrochloride, cholecalciferol, fur-sultiamine, inositol, derivatives thereof and mixtures thereof.

2. The ubidecarenone-containing multi-layered vitamin complex tablet according to claim 1, wherein the ubidecarenone content is maintained at 90% or more over the period of a 24 months long-term stability test performed at 25±2° C. and 60±5% RH, or a 6 months accelerated stability test performed at 40±2° C. and 75±5% RH.

3. The ubidecarenone-containing multi-layered vitamin complex tablet according to claim 1, wherein the second layer further contains at least one active ingredient selected from the group consisting of:
a mineral selected from zinc sulfate, iron sulfate, choline bitartrate, calcium hydrogen phosphate and mixtures thereof;
an amino acid selected from L-cysteine, derivatives thereof and mixtures thereof; and
a herbal extracts comprising γ-oryzanol, derivative thereof or mixtures thereof.

4. The ubidecarenone-containing multi-layered vitamin complex tablet according to any one of claims 1 to 3, wherein the first and the second layers together forms a structure of upper/lower layers or inner/outer layers.

5. The ubidecarenone-containing multi-layered vitamin complex tablet according to claim 4, wherein the second layer is divided into two separate sublayers, each of said sublayers comprises vitamin and mineral respectively, thereby the multi-layered tablet is in the form of three-layered tablet.

6. The ubidecarenone-containing multi-layered vitamin complex tablet according to claim 1, wherein the multi-layered tablet further comprises at least one selected from the group consisting of excipients, disintegrants, binders, glidants and sweetening agents.

7. The ubidecarenone-containing multi-layered vitamin complex tablet according to claim 1, wherein at least one of a moisture-proof coating and a light-shielding coating are applied to the multi-layered tablet.

8. A method for preparing an ubidecarenone-containing multi-layered vitamin complex tablet, comprising the steps of:
compressing one of raw materials for a first layer and for a second layer to form a compressed layer, said first layer containing ubidecarenone and said second layer containing at least one active ingredient, as a second ingredient, said active ingredient decreasing the stability of ubidecarenone; and
compressing the other raw material onto the compressed layer to form a structure of upper/lower layers or inner/outer layers to obtain a multi-layered tablet;
wherein the second ingredient contains a vitamin selected from the grouped consisting of ascorbic acid, alpha-tocopherol acetate, fursultiamine, retinol palmitate, cyanocobalamine, nicotinamide, pyridoxine hydrochloride, chole-calciferol, inositol, derivatives thereof and mixtures thereof.
